(19) United States
(12) Patent Application Publication
(10) Pub. No.: US 2008/0146669 A1
(43) Pub. Date: Jun. 19, 2008

(54) Novel Chloride Channel Pore Openers

(75) Inventors: Wei Wang, Birmingham, AL (US); Ge Li, Birmingham, AL (US); John P. Clancy, Homewood, AL (US); Kevin L. Kirk, Birmingham, AL (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(73) Assignee: THE UAB RESEARCH FOUNDATION, Birmingham, AL (US)

(21) Appl. No.: 11/815,413

(22) PCT Filed: Feb. 2, 2006
(86) PCT No.: PCT/US2006/003698

§ 371 Date: Aug. 2, 2007

Related U.S. Application Data
(60) Provisional application No. 60/649,386, filed on Feb. 2, 2005.

(51) Int. Cl. A61K 31/12; A61K 31/195; A61P 11/00

(52) U.S. Cl. 514/567; 514/679

(57) ABSTRACT

Described is a method for treating and/or preventing disease states and conditions associated with defects in the activity of chloride channels, such as the CFTR channel. The present disclosure also described compounds useful in such treatment and prevention methods and methods for the identification of compounds for use in such methods. These compounds disclosed are NPPB, NPPB-Am, NPPB-sulf and curcumin, and derivatives of the foregoing. These compounds are shown to activate the activity of both wild-type and mutant CFTR channels under a range of conditions.

### FIELD OF THE DISCLOSURE

The present disclosure relates generally to methods of treatment and/or prevention of a disease state or condition associated with a defect in the activity of a chloride channel, such as, but not limited to, the CFTR channel, and the identification of compounds useful in such methods of treatment and/or prevention. The present disclosure also relates to methods of activating chloride channels and to the identification of compounds for use in such methods of activation.

### BACKGROUND

Chloride channels play essential roles in controlling membrane potential, cell volume and salt transport in all tissues and organs. A variety of human disease states and conditions are caused by or associated with defects in the function of chloride channels. These defects include, but are not limited to, defects in chloride channel activity. Exemplary disease states associated with defects in chloride channel function include, but are not limited to, cystic fibrosis (CF), myotonia, deafness and kidney disease (for example, Bartter's syndrome). CF alone affects approximately 80,000 individuals.

CF is a lethal genetic disease caused by a mutation in a membrane protein, the cystic fibrosis transmembrane conductance regulator (CFTR), which functions primarily as a chloride channel. Defects in the activity of the CFTR are associated with decreased secretion of Cl-from the epithelial cells lining the airways of the lungs, resulting in a mucous lining that is abnormally thick due to decreased hydration. The normal function of the mucous lining of the lungs serves to trap particles, bacteria and other harmful organisms and aid in clearing these agents from the lungs (cilia present in the epithelial cells normally clear the bacteria trapped in the mucous lining). In individuals without CF, the mucous lining is maintained in a diluted state due to the transport of Cl-ions from the epithelial cells which results in hydration of the mucous lining. The thicker mucous lining in individuals affected with CF cannot be cleared from the lungs efficiently. This results in the agents trapped by the mucosal lining remaining in the lungs where such agents can serve as a locus for inflammation and infection. Additionally, the thick mucous lining provides additional binding sites for bacteria that progressively destroy the airway tissue. The consequence is a dry, infected ‘cystic fibrotic’ lung. Defects in the activity of the CFTR are also associated with malabsorption and maldigestion of food and nutrients from the intestine due to a failure of enzyme secretion by the pancreatic ducts and intestinal blockage (meconium ileus) due to the failure of fluid secretion by the intestinal crypt epithelium. Other affected tissues include the sweat gland, where the sweat duct cannot reabsorb Cl- and hence dilute the sweat; the liver; and the ovaries, uterus and vas deferens of the testes, which leads to infertility in both affected males and females.

Although many mutations in the CFTR channel have been associated with CF, the most common CF mutant is ΔF508 CFTR. In this mutation a single phenylalanine residue is deleted from position 508 of the CFTR. The ΔF508 mutation results in the inefficient transfer of the CFTR protein to the apical membrane of the epithelial cells lining the airways and the crypt regions of the small and large intestines. However, the ΔF508 mutation has been argued to have near-normal ATP-dependent gating (i.e. channel opening and closing) when it reaches the apical membrane (2, 3; although see 4).

The development of drugs to treat disease states that are caused by defects in chloride channel activity is in its infancy. As an example, several strategies for combating defects in the CFTR have been developed. One approach is to inhibit the degradation of mutant CFTR proteins (such as ΔF508) by targeting the cellular components responsible for degradation of the mutant CFTR proteins. As a result, the mutant CFTR proteins are trafficked to the cell membrane. An alternate approach is to compensate for the defective chloride transport by CFTR by stimulation of other chloride channels via activation of purinergic receptors. A further approach is to maintain hydration of the airway mucus by inhibiting sodium uptake by the epithelial sodium channel using amiloride. Clinical tests so far have been inconclusive (23). Clearly, additional treatment methodologies for use in treating disease states associated with abnormal chloride channel activity are needed. The present disclosure provides new methods of treatment and/or prevention and identifies candidate compounds for use in the disclosed methods.

In this disclosure, methods for treatment and/or prevention of a disease state or condition associated with defects in the activity of chloride channels, such as the CFTR, are provided. Such methods may be used to treat and/or prevent a disease state or condition caused by defects in the activity of a chloride channel, such as the CFTR, or to treat and/or prevent or a disease state or condition associated with defects in the activity a chloride channel, such as the CFTR. Several classes of compounds are described that maybe used in such treatment and prevention methods. In addition, methods for the identification of compounds that may be used in the methods of treatment disclosed are provided. Methods for the activation of chloride channels, such as the CFTR, are also disclosed.

### BRIEF DESCRIPTION OF THE FIGURES

FIGS. 1A-F show that NPPB behaves as a mixed agonist toward thiolated and poorly phosphorylated CFTR channels.

FIG. 1A shows that NPPB (200 μM) stimulates positive currents mediated by thiolated CFTR channels in excised BHK-CFTR (baby hamster kidney cells stably expressing wild type human CFTR) patches. CFTR was activated with 110 U/ml PKA (high PKA) and 1.5 mM MgATP. Further phosphorylation was blocked by the addition of 1.4 μg/ml PKI. The moderate decrease in current following PKI addition is presumably due to the activity of membrane-bound phosphatases. Equimolar diamide/glutathione (GSH) (20 μM) was added to promote channel thiolation (7). See Methods Section for ramp protocol and other technical details.

FIG. 1B shows that a derivative of NPPB that lacks the benzamide ring (APB) fails to stimulate thiolated channels at 400 μM under the same conditions as tested in FIG. 1A.

FIG. 1C shows that NPPB (30-230 μM) markedly stimulates currents mediated by poorly phosphorylated CFTR channels that have not been thiolated. “Low PKA” refers to 2 U/ml PKA.

FIG. 1D shows that NPPB blocks both positive and negative currents for highly phosphorylated CFTR channels that have not been thiolated. “Highly phosphorylated” refers to high PKA without the addition of PKI; (Glib denotes glibenclaminde, a voltage dependent blocker of the CFTR pore.

FIG. 1E shows that NPPB causes voltage-dependent block at all doses (5-300 μM) for moderately phosphorylated wild-type channels. “Moderately phosphorylated” refers to high PKA followed by PKI. The result shown in representative of seven experiments.

FIG. 1F shows that at low doses, NPPB stimulates currents in both directions for moderately phosphorylated R347D-CFTR channels in an excised patch from a transiently transfected HEK-293T cell. The conditions used were identical to those in FIG. 1E. The result shown in representative of five experiments.

FIGS. 2A-F show CFTR channel activation by a neutral NPPB analog and by curcumin.

FIG. 2A shows the chemical structures of tested compounds.

FIG. 2B shows that NPPB-AM (the neutral NPPB analog) at 10 μM stimulates currents in both directions for moderately phosphorylated wild-type channels in excised BHK-CFTR patches. Results are representative of 6 experiments. Also see mean data for HEK-293T cells in FIG. 4B.

FIG. 2C shows NPPB-Am titration for a representative patch. The conditions used were the same as those described in FIG. 1B. Results were obtained for the same patch, and are representative of 3 experiments. The lowest dose tested was 250 nM, which increased the current by 25%.

FIG. 2D show the mean titration data fit to the Michaelis-Menten function (EC₅₀=0.96+0.19 μM). The conditions were as described for B and C. Data were normalized to the peak current at 10 μM NPPB-AM. The lowest dose tested was 125 nM, which increased the current by approximately 20%. The results are means±S.E. (n=8).

FIG. 2E shows that NPPB-Am has a greater relative stimulatory effect on poorly phosphorylated CFTR channels.

FIG. 2F shows that curcumin (0.5-40.5 μM)) also stimulates moderately phosphorylated CFTR channels in an excised BHK-CFTR patch. Results are representative of six experiments.

FIGS. 3A-F show that NPPB-Am increases the rate of CFTR channel opening without affecting CFTR phosphorylation.

FIG. 3A shows that NPPB-Am (10 μM) and NPPB (50 μM) markedly stimulate the currents in excised HEK-293T patches mediated by AR-S660A-CFTR in the absence of PKA. The dotted line indicates change in scale. Results are representative of 3 experiments.

FIG. 3B shows that neither NPPB-Am nor NPPB affects currents across a patch excised from an untransfected (CFTW) HEK-293T cells.

FIG. 3C shows that NPPB-Am (10 μM) stimulates the opening rate of poorly phosphorylated CFTR channels. Data were obtained using a BHK-CFTR ‘micropatch’ that contained 8 detectable channels after NPPB-Am addition. Patches were held at −80 mV. Channels were first activated by 2 units/ml PKA, followed by PKI. Pₒ and opening rates per channel were estimated from 3-5 min records assuming 8 active channels for each condition. Results are representative of 3 experiments.

FIG. 3D shows mean data illustrating that NPPB-Am stimulates the opening rates of poorly phosphorylated CFTR channels. The conditions were as described for C. The data are means±S.E. (n=7). Opening rates represent the total number of openings/s/patch. The “best guess” estimates of the mean single channel opening rates and single channel Po values for the pre- and post-NPPB-Am conditions (assuming that Nis the maximum number of simultaneous openings after NPPB-Am addition) were as follows: 0.26±0.09 (pre) and 1.07±0.29 (post) openings/s/channel and Pₒ=0.06±0.01 (pre) and 0.26±0.06 (post) (means±S.E., n=7).

FIG. 3E shows that NPPB-Am is a weak activator of G551D-CFTR channels at 10 μM whereas high doses of NPPB (200 to 400 μM) can markedly stimulate this mutant. Data were obtained from HEK-293T cells. Dotted line indicates scale change. Mean data can be seen in FIG. 3E.

FIG. 3F shows that NPPB stimulates wild type CFTR at lower doses than for G551D-CFTR channels. Constructs were expressed in HEK-293T cells. Wild type channels were activated with low PKA. The conditions for G551D were the same as those described in FIG. 3D. Also shown are mean data at two voltages obtained from 4 and 7 experiments for wild-type and G551D, respectively. Data were normalized to peak currents induced by NPPB at +80 mV and −80 mV.

FIGS. 4A-E show that NPPB-AM (10 μM), NPPB (100 μM) and curcumin (10 μM) markedly stimulate the activities of ΔF508-CFTR channels.

FIG. 4A shows stimulation of macroscopic ΔF508-CFTR currents in excised HEK-293T patch by NPPB-Am (10 μM), curcumin (10 μM) and NPPB (100 μM). ΔF508-CFTR was ‘temperature-corrected’ by growing the cells at 28° C. for 24 hrs. Channels were exposed to high PKA (110 U/ml) without PKI for 5 min prior to the addition of compound.

FIG. 4B shows mean data showing that NPPB-AM much more greatly stimulates ΔF508-CFTR currents than wild-type currents (WT) in excised patches exposed to a high PKA concentration (110 U/ml) and 1.5 mM MgATP. PKI, when added, was added at a concentration of 1.4 μg/ml Error bars indicate the means±S.E. of five to eight experiments. All results were obtained from wild-type CFTR- or ΔF508-CFTR-transfected HEK-293T cells with the exception of the gray bar (CFBE41o⁻cells stably transfected with ΔF508-CFTR, (19)). AF508-CFTR-expressing cells were “low temperature-corrected” as described.

FIG. 4C shows a micropatch recording demonstrating a large stimulation of Pₒ and opening rate for ΔF508-CFTR by NPPB-Am (10 μM). Channels were exposed to high PKA, 1.5 mM MgATP, and held at a membrane potential of −80 mV. For this patch there were sufficiently few simultaneous openings (seven) after NPPB-Am addition to allow estimates of Pₒ and opening rates for both conditions. N was assumed to be 7 for both conditions for the analysis, although fewer simultaneous openings were detected prior to adding the compound. The results are representative of 4 experiments.

FIG. 4D shows effects of NPPB-Am on the mean opening rates and Po for ΔF508-CFTR in excised membrane patches (n=four HEK-293T patches). Opening rates represent the total number of openings/s/patch. The best guess estimates of the mean single channel opening rates and single channel Po values for the pre-and post-NPPB-AM conditions (assuming that N is the maximum number of simultaneous openings after NPPB-AM addition) were as follows: 0.20±0.11 (pre) and 0.87±0.31 (post) openings/s/channel and Po=0.05±0.03 (pre) and 0.25±0.07 (post) (mean±S.E., n=4).

FIG. 4E shows a representative experiment demonstrating a dramatic increase in ΔF508-CFTR channel activity in excised HEK-293T following NPPB-Am (10 μM) addition. Channels were exposed to high PKA, 1.5 mM MgATP, and held at a membrane potential of −80 mV. Although only 2 channels were detected prior to NPPB-Am addition, the patch contained at least 12-15 channels based on the large increase in glibenclamide-sensitive current that was induced by NPPB-Am. Pₒ and opening rate per channel were estimated for the control condition assuming two active channels in the patch (n=2).

FIGS. 5A-B show that NPPB-Am stimulates AF508-CFTR currents in intact CFBE41o⁻bronchial epithelial monolayers.

FIG. 5A shows the effect of NPPB-Am on CFBE41o- bronchial epithelial cells stably transfected with ΔF508-CFTR and cultured as electrically resistive monolayers at low temperature and assayed in Ussing chambers. A serosal-to-mucosal Cl⁻ gradient (120 mM to 1.2 mM) was imposed followed by addition of 100 μM amiloride to block Na⁺ currents. NPPB-Am (0.12-30 μM) and glibenclamide (400 μM) were added to both chambers at the indicated concentrations. A 3 mV voltage pulse was imposed every 100 sec to estimate transepithelial resistance.

FIG. 5B shows that low dose NPPB-Am (1 μM) potentiates ΔF508-CFTR activation by low dose forskolin (1 μM) in CFBE41o- monolayers whereas genistein (1 μM) does not. The conditions were the same as those described in FIGS. 5A and 5B. N=8-12 for each treatment. * p=0.001 by Mann-Whitney test (vs. genistein+forskolin).

### DETAILED DESCRIPTION

### Definitions

The terms “prevention”, “prevent”, “preventing”, “prevented”, “suppression”, “suppress”, “suppressing” and “suppressed” as used herein refer to a course of action (such as administering a compound or pharmaceutical composition) initiated prior to the onset of a symptom, aspect, or characteristics of a disease state or condition so as to prevent or reduce said symptom, aspect, or characteristics. Such preventing and suppressing need not be absolute to be useful.

The terms “treatment”, “treat” and “treating” as used herein refer to a course of action (such as administering a compound or pharmaceutical composition) initiated after the onset of a symptom, aspect, or characteristics of a disease state or condition so as to eliminate or reduce said symptom, aspect, or characteristics. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, as the result of a disease state or condition that is treatable by a method or compound of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a disease state or condition that is preventable by a method or compound of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as contained in a pharmaceutical composition that is capable of having any detectable, effect on any symptom, aspect, or characteristics of a disease state or condition. Such effect need not be absolute to be beneficial.

The term “disease state” as used herein refers to any pathological condition of a cell, a body part, an organ, a tissue or a system resulting from a cause.

The term “condition” as used herein refers to any manifestation, symptom, disorder or state associated with a disease state.

### General

In the present disclosure, methods of treating and/or preventing a disease state or condition associated with defects in the activity of a chloride channel, such as the CFTR channel, are provided. The present disclosure also provides exemplary compounds that may be used in the treatment and/or prevention methods described. In addition, methods of screening are provided to identify compounds that may be useful in the methods of treatment and/or prevention disclosed. Furthermore, the present disclosure also provides for methods of activating the chloride transport mediated by chloride channels, compounds useful in such methods o activation and methods for the identification of additional compounds that may be useful in such methods of activation.

The present disclosure described several classes of compounds that stimulate the activity of chloride channels using the CFTR channel as an exemplary chloride channel (25). The stimulation shown includes both wild-type and mutant CFTR channels, such as the AF508 CFTR. The compounds disclosed include sulphonylureas, charged (NPPB) and neutral (NPPB-Am) arylaminobenzoate derivatives and curcumin; as used herein, reference to any of the foregoing compounds also includes a modification to the compound. As used herein, a modification shall include pharmaceutically acceptable salts thereof, esters thereof, prodrugs thereof or tautomers thereof as well as polymorphic variants of any of the foregoing. The present disclosure utilizes the CFTR as an exemplary chloride channel and cystic fibrosis as an exemplary disease state for the purposes of illustrating the many aspects of the methods and compounds disclosed. However, the methods and compounds of the present disclosure should not be limited to the CFTR or to cystic fibrosis. One of ordinary skill in the art would realize that the compounds and methods disclosed herein will have utility beyond application to the CFTR and cystic fibrosis.

Charged compounds in these classes are shown to have mixed agonistic and antagonistic activities with respect to CFTR activity (i.e., stimulatory at low concentrations and inhibitory at high concentrations). An uncharged arylaminobenzoate derivative, NPPB-Am, and curcumin are shown to have pure agonistic activity at all concentrations examined. All of the compounds disclosed are cell permeant and can readily enter cells where they act to enhance chloride channel activity. Each compound can also activate CFTR channels under a wide variety of CFTR channel modification conditions, including conditions under which this channel is normally closed. For example, the compounds disclosed can stimulate CFTR channels that are poorly phosphorylated or that have been oxidized by glutathionylation (conditions which are normally associated with reduced CFTR channel activity).

The uncharged arylaminobenzoate derivative, NPPB-Am, and a dietary compound curcumin are shown in this disclosure to stimulate CFTR opening without blocking the pore. These compounds dramatically stimulate the opening of membrane-resident ΔF508 CFTR channels under conditions when the wild type channel is nearly maximally active. These data indicate that the ΔF508 mutation substantially disrupts CFTR channel gating, and that the activity of this mutant channel is greatly enhanced by the compounds disclosed. This gating defect became apparent when after increasing channel opening rate with NPPB-Am, it became clear that the number of ΔF508-CFTR channels had been substantially underestimated under control conditions. This observation is important since the prevailing view is that mutant CFTR channels possessed near normal activity once they reach the cell membrane. The present shows that certain mutant CFTR channels, such as the ΔF508 CFTR channel, have decreased activity and that activity can be restored through therapeutic intervention with appropriate compounds using the methods of treatment and prevention as disclosed herein.

The therapeutic potential of these compounds is further supported by the fact that a number of chemically similar sulfonylureas have already been approved by the FDA for the treatment of a variety of human disorders. Curcumin is a dietary compound, suggesting its administration is safe. The compounds described have several additional features that make them attractive candidates for drug development, including, but not limited to, specificity, simple chemistry and synthesis and ability to cross cell membranes. Therefore, the compounds disclosed maybe useful in the treatment of disease states and conditions associated with defects in the activity of chloride channels, such as the CFTR channel. Such disease states may include CF and conditions associated with CF such as, but not limited to, respiratory infections and lung complications, vitamin deficiencies, malnutrition, malabsorption, pancreatitis, diabetes, meconium ileus, weight loss, failure to thrive, delayed growth, delayed sexual development at puberty, growths (polyps) in the nasal passages, enlargement or rounding (clubbing) of the fingertips and toes, coughing or wheezing, thick sputum, biliary cirrhosis caused by blocked bile ducts in the liver, rectal prolapse.

### Methods

Methods for the treatment and prevention of disease states and conditions associated with defects in the activity of chloride channels, such as the CFTR are disclosed. Furthermore, methods for increasing the activity of such channels are disclosed. In addition, compounds and methods for identifying novel compounds useful in the methods disclosed are described. The present disclosure describes in detail the application of these teachings to the methods of treatment and/prevention of disease states and conditions associated with defects in CFTR channel activity using CF as a model.

The present disclosure provides a method of treating a disease state or condition associated with defects in the activity of chloride channels, such as the CFTR. In one embodiment, the method comprises the steps of: (i) identifying a subject who is suffering from a disease state or condition associated with defects in the activity of chloride channels, such as the CFTR and who is in need of treatment; (ii) administering a therapeutically effective amount of a compound (or pharmaceutical composition comprising such compound) to the subject so that the disease state or condition is treated. In one embodiment, the method of treatment restores, at least partially, the normal activity of the chloride channel. In a specific embodiment the chloride channel is the CFTR and the disease state if CF, or a condition associated with CF. In one embodiment, the method of prevention restores, at least partially, the normal activity of the CFTR.

The present disclosure also provides a method of preventing a disease state or condition associated with defects in the activity of chloride channels, such as the CFTR. In one embodiment, the method comprises the steps of: (i) identifying a subject who is at risk for a disease state or condition associated with defects in the activity of chloride channels, such as the CFTR and who is in need of prevention; (ii) administering a therapeutically effective amount of a compound (or pharmaceutical composition comprising such compound) to the subject so that the disease state or condition is prevented. In one embodiment, the method of prevention restores, at least partially, the normal activity of the chloride channel. In a specific embodiment the chloride channel is the CFTR and the disease state if CF, or a condition associated with CF. In one embodiment, the method of prevention restores, at least partially, the normal activity of the CFTR.

The compound administered may be any of the compounds described herein (sulphonylureas, arylaminobenzoates and curcumin, and derivatives of the foregoing) or any compound identified by the screening methods described herein. The compound may be administered alone or in a suitable pharmaceutical composition. The compound or pharmaceutical composition comprising the compound may be formulated by any method known in the art. Certain exemplary methods for preparing the compounds and pharmaceutical compositions are described herein and should not be considered as limiting examples. Furthermore, the compounds or pharmaceutical compositions containing the compounds may be administered to the subject as is known in the art and determined by a healthcare provider. Certain modes of administration are provided herein and should not be considered as limiting examples. Furthermore, the compound or pharmaceutical composition maybe administered with other agents in the methods described herein. Such other agents maybe agents that increase the activity of the compounds disclosed, such as by limiting the degradation or inactivation of the compounds disclosed, increasing the absorption or activity of the compounds disclosed or treating and/or preventing other aspects of the disease state of condition.

In one embodiment, the compound (whether administered alone or as a part of a pharmaceutical composition) treats and/or prevents the disease state or condition through activation of a chloride channel such as the CFTR channel. This channel activation may be a direct activation of CFTR channel activity. In one embodiment, the direct activation occurs by binding of the compound to CFTR. Channel activation may also be an indirect activation. In either case, the compound (whether administered alone or as a part of a pharmaceutical composition) is administered in a therapeutically effective amount.

In another embodiment of the present disclosure, there is provided a method for identifying compounds that are useful in the methods of treatment and/or prevention describes or that modulate the activity of chloride channels, such as the CFTR channel. The present disclosure provides methods of screening compounds to achieve such purposes. In one embodiment, the method comprises the steps of: (i) providing a test system expressing a chloride channel (such as the CFTR or a mutant thereof); (ii) providing a compound; (iii) treating the test system with the compound; and (iv) identifying compounds that modulate the activity of the chloride channel by determining the activity of the chloride channel in the test system in the presence and/or absence of the compound.

The methods for determining the activity of chloride channels and the CFTR specifically are known in the art. Any such method may be used in the methods disclosed herein. Furthermore, certain methods are described herein. In one embodiment, the compounds may increase the activity of the chloride channel. In an alternate embodiment, the compounds may decrease the activity of the chloride channel. In one embodiment, the chloride channel in the test system are provided in a functional state. A functional state is defined as a chloride channel, alone or in combination with other components (such as those required for the regulation of the chloride channel) such that the chloride channel is active to at least some measurable degree either before, during or after administration of the compound to be tested. The chloride channel in the functional state may be a wild-type channel or may contain mutations. With regard to the CFTR, any of the mutant CFTR forms may be used. The chloride channel may be recombinantly expressed if desired. The test system may utilize a cell line, a cell free preparation, a preparation derived from a cell, such as a cell patch, or a cell monolayer, oocytes, lipid bilayers, mammalian, drosophila, bacterial or yeast cells. Furthermore, membrane preparations or vesicles can be formed from any of the above and used to conduct the identification procedures and used as the test system.

The activation of chloride channels, such as the CFTR channel, maybe determined directly or indirectly. In one embodiment of direct determination, the chloride currents generated in response to the compound being tested may be determined by methods known in the art and/or as described herein. Other methods of direct determination may be used. In one embodiment of indirect determination, the binding of the compounds to the chloride channels in the test system may be determined or the degree of a tissue pathology, such as, but not limited to, the measurement of markers of inflammation or infection, may be determined. Other methods of direct or indirect determination may also be used. The compounds to be tested may be labeled or may be conjugated to a detection molecule as is known in the art. Such detection molecules are well known in the art, including, but not limited to, a radiolabel, a light-emitting label, a fluorescent label or an enzymatic label.

The present disclosure provides compounds which increase the activity of chloride channels, such as the CFTR, and methods for increasing such activity. In one embodiment, the method for increasing the activity of a chloride channel, such as the CFTR channel, comprises the steps of; (i) providing a test system, cell or subject expressing a chloride channel (such as the CFTR) and (ii) providing a compound in an amount effective to increase the activity of the chloride channel. The compound administered may be any of the compounds described herein (sulphonylureas, arylaminobenzoates and curcumin, and derivatives of the foregoing) or any compound identified by the screening methods described herein. In one embodiment, the subject is a human subject.

The compounds are shown to increase the activity under a wide range of CFTR modifications. Therefore, the compounds described are expected to have clinical utility in treating and/or preventing a variety of disease states and/or conditions associated with defects in the activity of chloride channels, such as the CFTR. For example, such disease states and conditions may include CF and conditions associated with CF such as, but not limited to, respiratory infections and lung complications, vitamin deficiencies, malnutrition, malabsorption, pancreatitis, diabetes, meconium ileus, weight loss, failure to thrive, delayed growth, delayed sexual development at puberty, growths (polyps) in the nasal passages, enlargement or rounding (clubbing) of the fingertips and toes, coughing or wheezing, thick sputum, biliary cirrhosis caused by blocked bile ducts in the liver, rectal prolapse.

The compounds of the present disclosure can be administered by any conventional method available for use in conjunction with pharmaceutical compositions. The compounds of the present disclosure may be administered alone or as a component of a pharmaceutical composition and may be administered with additional active agents if desired.

The compounds and pharmaceutical compositions described can be used in the form of a medicinal preparation, for example, in aerosol, solid, semi-solid or liquid form which contains the compounds disclosed as an active ingredient. In addition, the pharmaceutical compositions may be used in an admixture with an appropriate pharmaceutically acceptable carrier. Such pharmaceutically acceptable carriers include, but are not limited to, organic or inorganic carriers, excipients or diluents suitable for pharmaceutical applications. The active ingredient may be compounded, for example, with the usual non-toxic pharmaceutically acceptable carriers, excipients or diluents for tablets, pellets, capsules, inhalants, suppositories, solutions, emulsions, suspensions, aerosols and any other form suitable for use. Pharmaceutically acceptable carriers for use in pharmaceutical compositions are well known in the pharmaceutical field, and are described, for example, in Remington: The Science and Practice of Pharmacy Pharmaceutical Sciences, Lippincott Williams and Wilkins (A. R. Gennaro editor, 20^{th} edition). Such materials are nontoxic to the recipients at the dosages and concentrations employed and include, but are not limited to, water, talc, gum acacia, gelatin, magnesium trisilicate, keratin, colloidal silica, urea, buffers such as phosphate, citrate, acetate and other organic acid salts, antioxidants such as ascorbic acid, peptides, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, lactose, mannitol, glucose, mannose, dextrins, potato or corn starch or starch paste, chelating agents such as EDTA, sugar alcohols such as mannitol or sorbitol, counterions such as sodium and/or nonionic surfactants such as Tween, Pluronics or polyethyleneglycol. In addition, the pharmaceutical compositions may comprise auxiliary agents, such as, but not limited to, taste-enhancing agents, stabilizing agents, thickening agents, coloring agents and perfumes.

Pharmaceutical compositions may be prepared for storage or administration by mixing a compound of the present disclosure having a desired degree of purity with physiologically acceptable carriers, excipients, stabilizers, auxiliary agents etc. as is known in the pharmaceutical field. Such pharmaceutical compositions may be provided in sustained release or timed release formulations.

The compound or pharmaceutical compositions containing the compound maybe administered orally in solid dosage forms, such as capsules, tablets, and powders, or in liquid dosage forms, such as elixirs, syrups and suspensions. Furthermore, compound or pharmaceutical compositions containing the compound may be administered parenterally, in sterile liquid dosage forms, by transmucosal delivery via solid, liquid or aerosol forms or transdermally via a patch mechanism or ointment. Various types of transmucosal administration include respiratory tract mucosal administration, nasal mucosal administration, oral transmucosal (such as sublingual and buccal) administration and rectal transmucosal administration.

For preparing solid compositions such as, but not limited to, tablets or capsules, the compounds described may be mixed with an appropriate pharmaceutically acceptable carriers, such as conventional tableting ingredients (lactose, sucrose, mannitol, corn starch, potato starch, alginic acid, microcrystalline cellulose, acacia, gelatin, gums, colloidal silicon dioxide, croscarmellose sodium, talc, sorbitol, stearic acid magnesium stearate, calcium stearate, zinc stearate, stearic acid, dicalcium phosphate other excipients, colorants, diluents, buffering agents, disintegrating agents, moistening agents, preservatives, flavoring agents, and pharmacologically compatible carriers) and diluents (including, but not limited to, water, saline or buffering solutions) to form a substantially homogenous composition. The substantially homogenous composition means the components (a compound as described herein and a pharmaceutically acceptable carrier) are dispersed evenly throughout the composition so that the composition maybe readily subdivided into equally effective unit dosage forms such as tablets, pills and capsules. The solid compositions described maybe coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact through the stomach or to be delayed in release. A variety of materials can be used for such enteric layers or coatings such materials including a number of polymeric acids and mixtures of polymeric acids with such materials as shellac, cetyl alcohol and cellulose acetate. The active compounds may also be formulated in rectal compositions such as suppositories or retention enemas, e.g., containing conventional suppository bases such as cocoa butter or other glycerides. The solid compositions may also comprise a capsule, such as hard- or soft-shelled gelatin type containing, for example, surfactants, lubricants, and inert fillers, such as lactose, sucrose, calcium phosphate, and corn starch.

For intranasal administration, intrapulmonary administration or administration by other modes of inhalation, the compounds disclosed (whether alone or in pharmaceutical compositions) may be delivered in the form of a solution or suspension from a pump spray container or as an aerosol spray presentation from a pressurized container or nebulizer, with the use of a suitable propellant (e.g., dichlorodifluoromethane, trichlorofluoromethane, dichlorotetrafluoroethane, nitrogen, propane, carbon dioxide or other suitable gas) or as a dry powder. In the case of an aerosol or dry powder format, the amount (dose) of the compound delivered may be determined by providing a valve to deliver a metered amount.

Liquid forms may be administered orally, parenterally or via transmucosal administration. Suitable forms for liquid administration include aqueous solutions, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. For buccal or sublingual administration, the composition may take the form of tablets or lozenges formulated in conventional manners. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds disclosed (whether alone or in pharmaceutical compositions) may be formulated for parenteral administration. Parenteral administration includes, but is not limited to, intravenous administration, subcutaneous administration, intramuscular administration, intradermal administration, intrathecal administration, intraarticular administration, intracardiac administration, retrobulbar administration and administration via implants, such as sustained release implants

The compounds disclosed (whether alone or in pharmaceutical compositions) may be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutically acceptable carriers for injectable compositions are well known to those of ordinary skill in the art.

The compounds disclosed (whether alone or in pharmaceutical compositions) are administered in a therapeutically effective amount. The therapeutically effective amount will, of course, vary depending upon known factors, such as the pharmacodynamic characteristics of the particular compound and its mode and route of administration; the age, health and weight of the subject; the severity and stage of the disease state or condition; the kind of concurrent treatment; the frequency of treatment; and the effect desired. The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

The following examples are given for the purpose of illustrating various embodiments of the methods and compounds of the instant disclosure and are not meant to limit the present disclosure in any fashion:

### EXAMPLES

### Example 1

### NPPB Behaves as a Mixed Agonist Toward Thiolated and Poorly Phosphorylated CFTR Channels

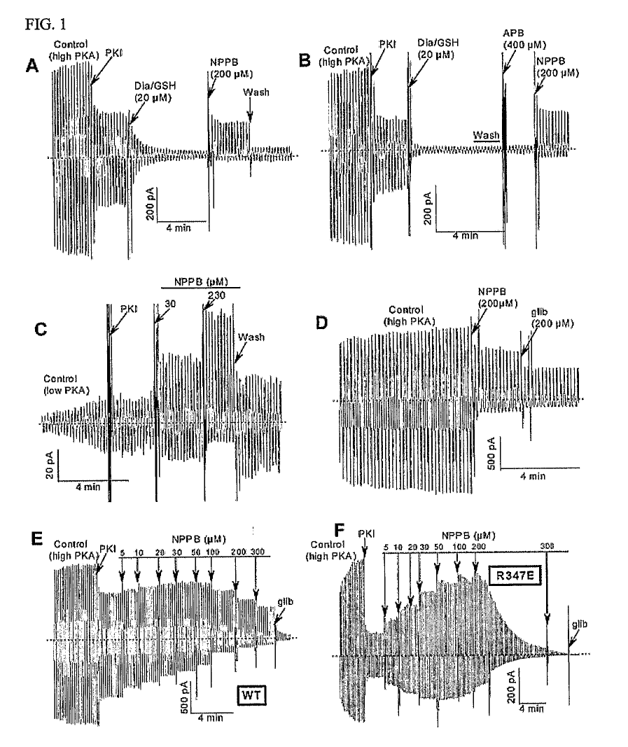

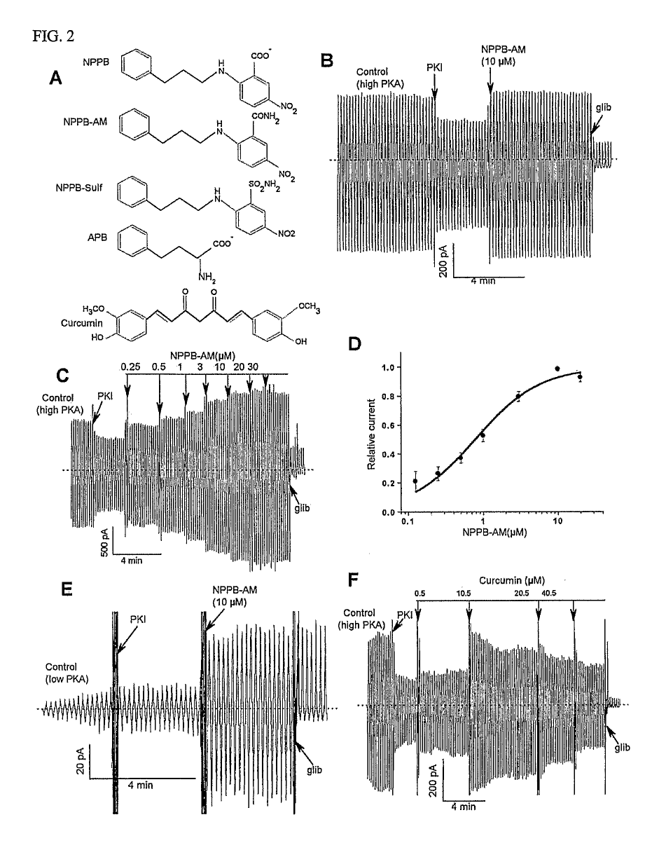

CFTR channel activation requires two events: (i) Mg-ATP binding to one or both nucleotide binding domains (NBDs) and (ii) phosphorylation of the regulatory (R) domain typically by protein kinase A (PKA) (5, 6). CFTR gating is further modulated by reactive glutathione species, which inhibit channel opening in the presence of Mg-ATP and PKA by glutathionylating a cysteine in the CFTR polypeptide (7 and FIG. 1A). A study on the effect of glutathionylation on CFTR gating led to the surprising finding that these modified channels could be activated by glibenclamide or NPPB, two negatively charged compounds that block the pore in a voltage-dependent manner. FIG. 1A shows the inhibitory effect of an equimolar mixture of glutathione (GSH) and diamide (a strong thiol oxidizer) on the macroscopic current mediated by many (>100) CFTR channels in an inside-out membrane patch excised from a CFTR-transfected BHK cell. This effect is due to glutathionylation of a cysteine in NBD2 (i.e., formation of a mixed disulfide with glutathione) that causes a dramatic reduction in channel opening rate (7). When 200 μM NPPB was added to block the residual CFTR current, a large increase in current at depolarizing voltages, voltages at which pore block by the negatively charged compound is less effective, was observed. Similar results were obtained for glibenclamide at 100-200 μM (not shown). The current that is induced by NPPB or glibenclamide at depolarizing potential is CFTR-mediated based on two criteria: (i) absent in membrane patches excised from CFTR-minus cells (see below and FIG. 2E) and (ii) inhibition at higher doses of blocker. The stimulatory effect of NPPB is specific in that neither 10 mM SCN⁻ or 0.5 mM DPC, other voltage-dependent blockers of the CFTR pore (8,9), or 400 μM APB, a truncated derivative of NPPB that lacks one of the aromatic rings (see structure in FIG. 2A), increased CFTR currents under these conditions (FIG. 1B).

Given the unexpected stimulatory effect of these pore blockers on glutathionylated channels, the effect of NPPB on the activity of unmodified (not glutathionylated) CFTR channels that are phosphorylated at low levels, that is, under conditions of submaximal stimulation by PKA, was determined. NPPB (30-230 μM) stimulated CFTR currents at depolarizing potentials when channels were first minimally phosphorylated by treating the patch with low PKA (2 U/ml) followed by PKA inhibitory peptide (PKI) to inhibit further phosphorylation (FIG. 1C). This stimulatory effect was observed at low micromolar concentrations of NPPB. At higher concentrations of NPPB, block was observed at all recording potentials. As expected, channels that were highly phosphorylated by continuous exposure to high PKA (110U/ml; standard CFTR activation conditions) failed to exhibit an appreciable increase in activity in response to NPPB at any voltage. Only voltage-dependent block was observed under these conditions (FIG. 1D). Thus, the relative enhancement of channel activity by NPPB is inversely related to the level of CFTR phosphorylation. This result shows that NPPB behaves as a mixed agonist toward thiolated or poorly phosphorylated CFTR channels (i.e. channels that are under sub-optimal stimulation), yet blocks the pore in a voltage-dependent fashion.

The effect of NPPB on the activity of the CFTR pore mutant R347D was then determined. The R347D mutant is resistant to pore block by NPPB (10). The rationale for this experiment was to determine if NPPB activates channels by binding to the same site that causes pore block or, alternatively, if the stimulatory effect of this partial agonist is exaggerated by mutating this site as would be expected if NPPB stimulates activity by binding to a different site. The results shown in FIGS. 1E and F suggest that NPPB stimulates channel activity by binding to a distinct site on the R347D CFTR channel. NPPB stimulated the currents mediated by R347D-CFTR at depolarizing potentials to a greater extent than for wild type CFTR at all levels of phosphorylation. In contrast to the wild type channel, R347D-CFTR currents were stimulated by NPPB even at hyperpolarizing potentials. These results show that NPPB stimulates channel opening by binding to a site that is distinct from the pore blocking site.

### Example 2

### NPPB-Am and Curcumin Activate CFTR Channels

The inhibitory effect of NPPB on CFTR currents presumably depends in part on the negative charge of this compound, as evidenced by the voltage-dependence of NPPB block. To identify compounds that are pure CFTR agonists, several neutral NPBB derivatives and other compounds were tested for their effects on CFTR channel activity; benzamide derivative of NPPB (NPPB-Am), a benzenesulfonamide derivative of NPPB (NPPB-sulf) and the dietary compound curcumin (see structures in FIG. 2A). NPPB-Am is a potent activator of CFTR channels. 10 μM NPPB-Am effectively stimulated the currents mediated by poorly phosphorylated or thiolated CFTR channels in excised membrane patches from BHK-CFTR cells (baby hamster kidney cells stably transfected with the wild-type human CFTR gene) (FIGS. 2B-D). NPPB-sulf had a weak effect at 100 μM (data not shown). The stimulatory effect of NPPB-AM was rapid, stable and quickly reversible upon wash out (FIG. 2B). The relative stimulation by the neutral NPPB-Am varied with the degree of CFTR phosphorylation with the greatest stimulatory effects observed at low levels of phosphorylation as was observed for the parent compound NPPB (compare FIGS. 2B and E). However, the stimulation by NPPB-Am was voltage-independent with no evidence for CFTR inhibition at any holding potential, unlike the case for the charged NPPB. Like the parent compound NPPB, NPPB-Am had no effect on currents across membrane patches excised from CFTR-minus cells (data not shown). In titration experiments detectable increases in CFTR currents were observed at NPPB-Am concentrations as low as 250 nM with an EC₅₀ of 0.71 μM (FIGS. 2C and 2D). Thus, NPPB-Am behaves like a pure CFTR agonist over the nanomolar to low micromolar concentration range.

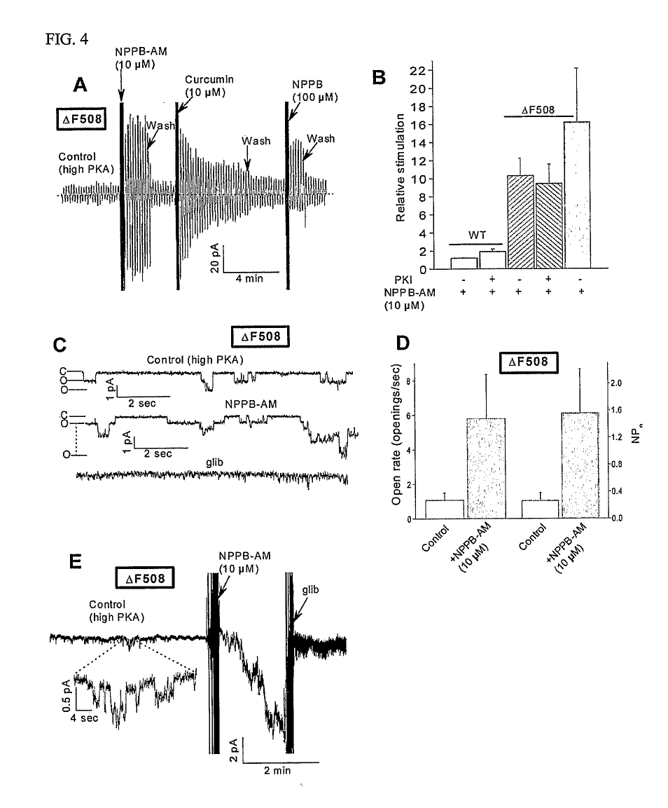

The structure of NPPB-Am (two aromatic rings separated by a hydrocarbon spacer) is generally similar to that of a dietary compound curcumin (a main ingredient in the spice turmeric; FIG. 2A). Egan, et al. (12) reported that curcumin promotes the biosynthetic maturation and functional correction of the AF508-CFTR mutant in tissue culture cells and in mice. The authors proposed that the mechanism for this apparent effect was indirect and involved perturbations in calcium pump activity and chaperone function in the endoplasmic reticulum (ER). Whether curcumin promotes the maturation of AF508-CFTR protein in the ER is controversial (13, 14). FIG. 2F shows that curcumin (0.5 to 10 μM) also stimulates the currents mediatedbywild-type CFTR in excised membrane patches from B K-CFTR cells. This stimulatory effect is voltage-independent as was observed for the neutral NPPB-Am. However, the stimulation of CFTR current by curcumin is transient, which is not a feature of CFTR activation by NPPB-Am. As will be described below, curcumin also stimulates AF508-CFTR channel activity (FIG. 4).

### Example 3

### NPPB-Am Increases the Rate of CFTR Channel Opening Independently of Any Effect of CFTR Phosphorylation

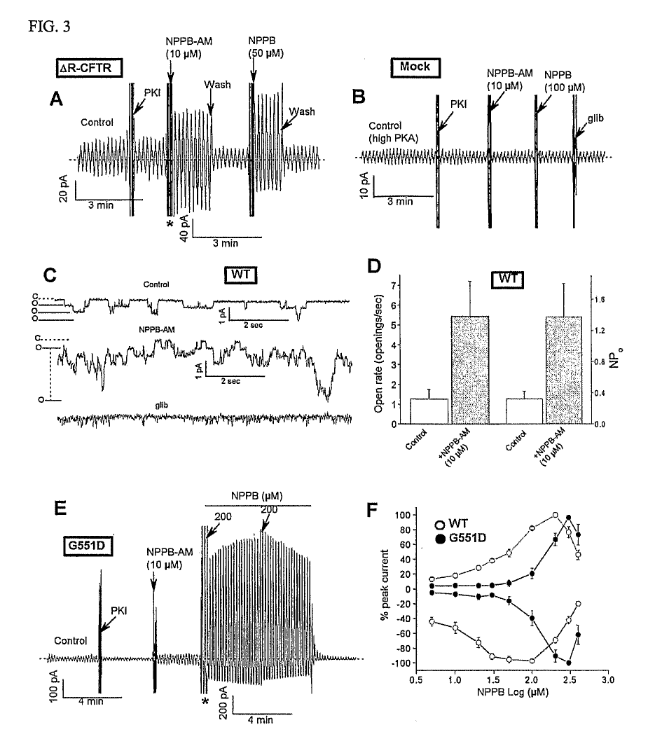

Other compounds have been reported to activate CFTR, but the mechanism by which they do so has not been determined. Possible mechanisms include a direct affect on CFTR channel gating, regulation of intracellular trafficking of CFTR (affecting channel number in the plasma membrane, or N) or signaling pathways that influence CFTR phosphorylation/dephosphorylation (15,16). FIG. 3 shows that the neutral NPPB derivative, NPPB-Am, stimulates the rate of CFTR channel opening in excised membrane patches (for which N is presumably constant) without affecting CFTR phosphorylation. Two lines of evidence indicate that NPPB-Am stimulates CFTR activity in excised patches without affecting the level of CFTR phosphorylation. First, NPPB and NPPB-Am reversibly stimulate CFTR currents following PKA wash out or the addition of PKI (FIGS. 1 and 2). Second, NPPB-Am markedly stimulated the currents mediated by an R domain deletion mutant (HEK-293 T cells expressing the AR-S660A-CFTR construct lacking the R domain which contains the regulatory sites of phosphorylation) that exhibits constitutive activity in the absence of PKA (FIG. 3A). Neither NPPB-Am or NPPB had any effect on HEK-293 T cells which where not transfected (FIG. 3B). NPPB-Am also had no obvious effect on the Mg-ATP sensitivity of CFTR activity as determined in Mg-ATP titration experiments performed in the presence and absence of this compound using patches obtained from BHK-CFTR cells (data not shown). On the other hand, NPPB-Am markedly increased the opening rates and mean single channel open probabilities (Pₒs) of poorly phosphorylated (2U/ml PKA) CFTR channels determined for ‘micropatches’ obtained from BHK-CFTR cells containing fewer than 10 channels each (FIGS. 3C and D).

Further evidence regarding the mechanism of NPPB-Am action can be seen in a study on the effects of NPPB and derivatives on the two most common CF mutants, G551D-CFTR and ΔF508-CFTR. G551D-CFTR is a well studied gating mutant (17) that, unlike ΔF508-CFTR, is trafficked to the cell surface with similar efficiency to wild-type CFTR. The G551D mutation maps to a region in NBD1 that likely plays a role in Mg-ATP binding or the conformational coupling between ATP binding and the opening of the pore within the transmembrane domains (ABC transporter signature sequence(18)). Interestingly, the activity of this mutant was only modestly stimulated by the neutral NPPB-Am at low micromolar concentrations (10 μM) as determined from HEK-293T cells expressing the G551D CFTR mutant (FIG. 3E). However, G551D-CFTR activity was markedly stimulated by high doses (200-400 μM) of the negatively charged parent compound NPPB, doses that were impossible to achieve for the less soluble uncharged derivative (FIG. 3E). In NPPB titration experiments an appreciable shift toward higher concentrations of NPPB for G551D-CFTR activation as compared to the wild type channel was observed (FIG. 3F). This result suggests that the G551D mutation in NBD1 reduces the apparent affinity of NPPB and of NPPB-Am for its activation site.

### Example 4

### NPPB-AM, NPPB and Curcumin Markedly Stimulate the Activities of Membrane Resident AF508-CFTR Channels

ΔF508-CFTR channels are dramatically stimulated by NPPB (100 μM), NPPB-Am (10 μM) and curcumin (10 μM) in excised patches from HEK-293 T cells expressing the ΔF508-CFTR channels (FIG. 4A). NPPB-Am (10 μM) induced an approximately 10-15-fold increase in ΔF508-CFTR macroscopic currents in excised patches from HEK-293T cells in the presence of normally saturating concentrations of 1.5 mM Mg-ATP and 110U/ml PKA (FIG. 4B). In FIG. 4B BHK denotes baby hamster kidney cells, HEK denotes HEK-293T cells and CFBE denotes CFBE41o- human bronchial epithelial cells cultured from a CF patient (19)). As observed for wild-type channel activity, NPPB-Am markedly stimulated F508-CFTR channel activity when added in the absence of active kinase (e.g. after adding PKI)(FIG. 4B). Similar results were obtained for ΔF508-CFTR channels that were expressed in CFBE41o- cells. This stimulation was due primarily to a large increase in channel opening rate as determined for ‘micropatches’ from HEK-293T cells expressing the ΔF508-CFTR channels and containing fewer than 10 detectable channels each (FIGS. 4C-D). This dramatic stimulation of ΔF508-CFTR activity by NPPB-Am occurred under conditions when the wild-type channel was nearly maximally active; the same dose of NPPB-Am stimulated the activity of the wild-type channel by only 1.5-2-fold (FIG. 4B).

The latter finding is inconsistent with the view that ΔF508-CFTR channels exhibit near-normal gating behavior when these channels reach the plasma membrane. This disparity can be explained, however, by recognizing the difficulty in estimating mean single channel Pₒ and opening rates for poorly active channels when the number of channels (N) in the patch is uncertain. FIG. 4E illustrates this point for a representative micropatch containing an unknown number of ΔF508-CFTR channels. Prior to the addition of NPPB-Am, no more than 2 simultaneous openings were observed. Based on this standard criterion for estimating N, the mean single channel Pₒ and opening rate per channel was initially calculated to be 0.04 and 1.2/sec, respectively. However, as many as 12-15 simultaneous openings were observed following the addition of NPPB-Am (assuming a unitary current of 0.5 pA/channel). Thus, it was clear that the number of channels in this patch had been underestimated for the control condition and, consequently, mean single channel Pₒ and opening rates had been grossly overestimated. These data suggest that ΔF508-CFTR channel opening is markedly inhibited under these experimental conditions, and argue that this gating defect in ΔF508-CFTR channels is normally difficult to appreciate because of uncertainties in estimating the numbers of channels in a membrane patch.

### Example 5

### NPPB-Am Stimulates ΔF508-CFTR Currents in Intact Epithelial Monolayers

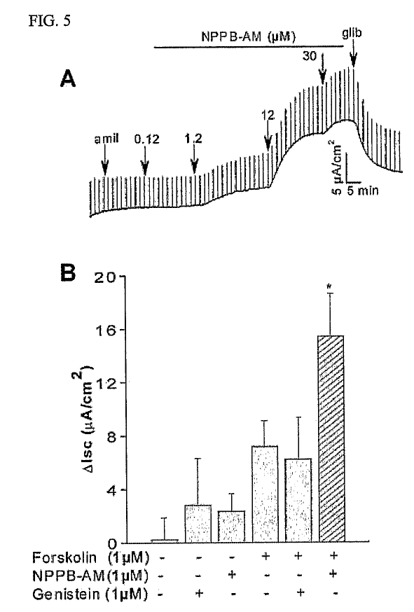

NPPB-Am also potently stimulates the activities of wild type and ΔF508-CFTR channels in intact epithelial cell monolayers. Parental CFBE41o -cells, which express undetectable levels of CFTR, and CFBE41o -cells that were transfected with ΔF508-CFTR or wild-type CFTR were grown as electrically resistive monolayers on permeable filters (19). ΔF508-CFTR-expressing monolayers were cultured at a reduced temperature (temperature-corrected) to enhance the surface expression of this mutant. CFTR-dependent transepithelial chloride currents were assayed in Ussing chambers (19). NPPB-Am had no effect on transepithelial currents assayed for parental CFBE41o- monolayers (1.1+/−2.7 μA/cm² increase in current; n=6) (data not shown). Conversely, NPPB-Am had two effects on the transepithelial chloride currents measured across wild type CFTR-expressing monolayers or temperature-corrected ΔF508-CFTR-expressing monolayers. First, at low doses (120 nM to 12 μM) this compound stimulated the transepithelial currents mediated by wild-type or ΔF508-CFTR channels without the simultaneous addition of a cAMP agonist (FIG. 5A). This stimulatory effect apparently required some level of constitutive CFTR phosphorylation by endogenous kinases, since it could be eliminated by prior treatment with the generic kinase inhibitor, H89 at 100 μM (5.5+/−1.3 μA/cm² (+H89) vs. 23.3+/−2.0 (−H89); n=4 for each condition) (data not shown). Secondly, 1 μM NPPB-Am markedly potentiated the effect of forskolin, an activator of cAMP synthesis, on the transepithelial currents mediated by ΔF508-CFTR (FIG. 5B). In contrast, genistein, a dietary compound previously reported to activate CFTR channels (20), failed to potentiate the forskolin response at this dose. These results indicate that NPPB-Am is a potent activator of wild type and ΔF508-CFTR channels in intact epithelial cell monolayers. The lack of effect of this compound on the transepithelial currents across CFTR-minus monolayers argues against a nonspecific effect of this compound on other types of anion channels in these cells.

In summary, potent CFTR agonists have been identified based on the surprising discovery that a pore blocker (NPPB) has mixed agonistic activity. The uncharged NPPB derivative (NPPB-Am) and the dietary compound curcumin behave as pure CFTR agonists that affect CFTR gating by increasing channel opening rate. The present disclosure also indicates that the ΔF508 mutation has a much greater effect on CFTR channel gating than initially appreciated. The literature on this point is confusing with reports of no gating defect for ΔF508-CFTR (3), a modest gating defect at high PKA (5, 6)) and a substantial gating defect (4). The results indicate that ΔF508-CFTR channels have at least 10-fold less activity (due in part to reduced opening rates) than wild type channels at normally saturating MgATP and PKA levels. This profound gating defect only became apparent after ΔF508-CFTR channel opening was stimulated by NPPB-AM, when it became clear that the numbers of ΔF508-CFTR channels under control conditions had been substantially underestimated. The markedly reduced channel activity of this mutant could exacerbate the severity of disease for ΔF508 patients especially if ΔF508-CFTR channels can reach the surfaces of a subset of epithelial cell types as argued by some (24). The present results support the emerging view that therapies which target only the biosynthetic processing defect of this mutant may be ineffective (6, 17).

The foregoing description illustrates and describes the methods and compounds of the present disclosure. Nothing herein is to be construed as an admission that the inventors are not entitled to antedate such disclosure by virtue of prior disclosure. Additionally, the disclosure shows and describes only certain embodiments of the methods and compounds but, as mentioned above, it is to be understood that the teachings of the present disclosure are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. All references cited herein are incorporated by reference as if fully set forth in this disclosure.

### Methods

For an additional description of the methods used in the present disclosure, see reference (25) and the references cited therein.

Cell culture DNA constructs and transfections. Baby hamster kidney (BHK) cells stably expressing wild type human CFTR (BHK-CFTR) were provided by J. Hanrahan (McGill University). BHK-CFTR cells were cultured in Dulbecco's modified Eagle's medium (DMEM (Mediatech)) supplemented with 5% fetal bovine serum (FBS) and 1 mM penicillin-streptomycin. The growth media for the BHK-CFTR cells also contained 0.5 mM methotrexate to maintain selection for CFTR-expressing cells (22). HEK-293T cells were transiently transfected with wild-type or mutant CFTR cDNA using the Lipofectamine transfection kit following manufacturer's recommendations (Invitrogen, Corp). HEK-293T cells were cultured in DMEM. All cells were grown on plastic coverslips for patch clamp recording and were used 2-3 days post-seeding.
Electrophysiology and data analysis. Macroscopic and multi-channel currents were recorded in the excised, inside-out configuration. Patch pipettes were pulled from Corning 8161 glass to tip resistances of 1.5-3.0 mOhm (macroscopic recordings) or 8-10 mOhm (micropatch studies). CFTR channels were activated following patch excision by exposure of the cytoplasmic face of the patch to catalytic subunit of protein kinase A (PKA; 110 U/ml; Promega) and Mg-ATP (1.5 mM). CFTR currents were recorded in symmetrical solution containing (in mM): 140 N-methyl-D-glucaniine-Cl, 3 MgCl₂, 1 EGTA and 10 TES. The pH was adjusted to 7.3. Macroscopic currents were evoked using a ramp protocol from +80 to −80 mV with a 10 sec time period. Patches were held at −80 mV or +80 mV for micropatch (multichannel) recordings. All patch clamp experiments were performed at 21-23° C. Signals from macroscopic and single channel recording were filtered at 20 and 200 Hz, respectively. Data acquisition and analysis were performed using pCLAMP8 and pCLAMP9 software (Axon Instruments). Curve fitting for kinetic analysis was performed using Microcal Origin software. Averaged data are presented as mean +/−SEM. Statistical comparisons were made by performing unpaired t-tests unless otherwise indicated.
Ussing Chamber Experiments-CFBE41o epithelial cells stably transfected with ΔF508-CFTR or transiently transfected with wild-type CFTR were cultured as electrically resistive monolayers and assayed in Ussing chambers as described (16). ΔF508-CFTR-expressing monolayers were grown at 27° C. for 2-3 days to enhance the surface expression of this temperature-sensitive mutant. A serosal-to-mucosal Cl⁻ gradient (120 to 1.2 mM) was imposed, followed by amiloride addition (100 μM) to block Na⁺ currents. Compounds were added to both chambers at the indicated concentrations. A 3-mV voltage pulse was imposed every 100 s to monitor transepithelial resistance.

### REFERENCES

1. Welsh, M. J. and A. E. Smith. Molecular mechanisms of CFTR chloride channel dysfunction in cystic fibrosis. Cell 73(7): 1251-1254, 1993.
2. Li, C., M. Ramjeesingh, E. Reyes, X. Chang, J. M. Rommens and C. E. Bear. The cystic fibrosis mutation (ΔF508) does not influence the chloride channel activity of CFTR. Nature Genetics 3:311-316, 1993
3. Wang, F., S. Zeltwanger, S. Hu and T.-C. Hwang. Deletion of phenylalanine 508 causes attenuated phosphorylation-dependent activation of CFTR chloride channels. J. Physiol. 524: 637-648, 2000.
4. Schultz, B. D., R. A. Frizzell and R. J. Bridges. Rescue of dysfunctional ΔF508-CFTR chloride channel activity by IBMX. J. Membrane Biol. 170: 51-66, 1999.
5. Gadsby, D. C. and A. C. Nairn. Control of CFTR channel gating by phosphorylation and nucleotide hydrolysis. Physiol. Rev. 79: S77-S 107, 1999.
6. Sheppard, D. N. and M. J. Welsh. Structure and function of the CFTR chloride channel. Physiol. Rev. 79:S23-S45,1999.
7. Wang, W., C. Oliva, G. Li, A. Holmgren, C. H. Lillig and K. L. Kirk. Reversible silencing of CFTR chloride channels by glutathionylation. J. Gen. Physiol. 125: 1-16, 2005.
8. Tabcharani, J. A., P. Linsdell and J. W. Hanrahan. Halide permeation in wild-type and mutant cystic fibrosis conductance regulator chloride channels. J. Gen. Physiol. 110:341-354, 1997.
9. Zhang, Z.-R., S. Zeltwanger and N. A. McCarty. Direct comparison of NPPB and DPC as probes of CFTR expressed in Xenopus oocytes. J. Membrane Biol. 175: 35-52, 2000.
10. Walsh, K. B., K. J. Long and X. Shen. Structural and ionic determinants of 5-nitro-2-(3-phenylpropyl-amino)-benzoic acid block of the CFTR chloride channel. Brit. J. Pharm. 127:369-376, 1999.
11. Bock, A., A. Krieger-Liszkay, I. Ortiz de Zarate and G. Schönknecht. Cl⁻ channel inhibitors of the arylaminobenzoate type act as photosystem II herbicides: a functional and structural study. Biochemistry. 40:3273-3281, 2001.
12. Egan, M. E., M. Pearson, S. A. Weiner, V. Rajendran, D. Rubin, J. Glockner-Pagel, S. Canny, K. Du, G. L. LuKacs and M. J. Caplan. Curcumin, a major constituent of turmeric, corrects cystic fibrosis defects. Science. 304: 600-602, 2004.
13. Song, Y., N. D. Sonawane, D. Salinas, L. Qian, N. Pedemonte, L. J. V. Galietta and A. S. Verkman. Evidence against the rescue of ΔF508-CFTR cellular processing by curcumin in cell culture and animal models. J. Biol. Chem. 279(39): 40629-40633, 2004.
14. Dragomir, A., J. Björstad, L. Hjelte and G. M. Roomans. Curcumin does not stimulate cAMP-mediated chloride transport in cystic fibrosis airway epithelial cells. Biochem. Biophys. Res. Commun. 322:447-451, 2004.
15. Marivingt-Mounir, C., C. Norez, R. Derand, L. Bulteau-Pignoux, D. Nguyen-Huy, B. Viossat, G. Morgant, F. Becq, J.-M. Vierfond and Y. Mettey. Synthesis, SAR, crystal structure and biological evaluation of benzoquinoliziniums as activators of wild-type and mutant cystic fibrosis transmembrane conductance regulator channels. J. Med. Chem. 47:962-972, 2004.
16. Yang H, Shelat A A, Guy R K, Gopinath V S, Ma T, Du K, Lukacs G L, Taddei A, Folli C, Pedemonte N, Galietta L J, Verkman A S. Nanomolar affinity small molecule correctors of defective Delta F508-CFTR chloride channel gating. J Biol Chem 278(37):35079-35085, 2003.
17. Gregory R J, Rich D P, Cheng S H, Souza D W, Paul S, Manavalan P, Anderson M P, Welsh M J, Smith A E. Maturation and function of cystic fibrosis transmembrane conductance regulator variants bearing mutations in putative nucleotide-binding domains 1 and 2. Mol & Cell Biol 11(8):3886-3893, 1991.
18. Higgins CF. ABC transporters: from microorganisms to man. Annu Rev Cell Biol 8:67-113, 1992.
19. Hentchel-FranksK, Lozano D, Eubanks-TarnV, Cobb B, FanL, OsterR, SorscherE, Clancy JP. Activation of airway Cl-secretion in human subjects by adenosine. Am J Resp Cell & Mol Biol 31(2):140-146, 2004.
20. Wang F, Zeltwanger S, Yang I C, Nairn A C, Hwang T C. Actions of genistein on cystic fibrosis transmembrane conductance regulator channel gating: Evidence for two binding sites with opposite effects. J Gen Physiol 111(3):477-490, 1998.
21. Dalemans W, Barbry P, Champigny G, Jallet S, Dott K, Dreyer D, Crystal R G, Pavironi A, Lecocq J P and Lazdunski M. Altered chloride ion channel kinetics associated with the delta F508 cystic fibrosis mutation. Nature 354:526-528, 1991.
22. Luo J, Pato M D, Riordan J R and Hanrahan J W. Differential regulation of single CFTR channels by PP2C, PP2A and other phosphatases. Am J Physiol 274:C1397-C1410, 1998.
23. Roomans, G M. Pharmalogical approaches to correcting the ion transport defect in cystic fibrosis. Am J Respir Med. 2003;2(5):413-31.
24. Kälin N, Claaβ, A, Sommer M, Puchelle E and Tümmler B. ΔF508 CFTR protein expression in tissues from patients with cystic fibrosis. J. Clin. Invest. 103(10): 1379-1389, 1999.
25. Wang W, Li G, Clancy J P and Kirk K. Activating Cystic Fibrosis Transmembrane Conductance Regulator Channels with Pore Blocker Analogs. J. Biol. Chem. 280(25): 23622-23630, 2005.

What is claimed:

1. A method for treating cystic fibrosis in a subject in need of such treatment, said method comprising the step of administering to said subject a therapeutically effective amount of a compound capable of stimulating the activity of a cystic fibrosis transmembrane conductance regulator.

2. The method of claim 1 where said compound is an arylaminobenzoate.

3. The method of claim 2 where said arylaminobenzoate has a negative charge.

4. The method of claim 2 where said arylaminobenzoate is NPPB.

5. The method of claim 4 where said NPPB is a chloride channel agonist at low concentrations and is a chloride channel antagonist at high concentrations.

6. The method of claim 1 where said compound is an arylaminobenzoate derivative.

7. The method of claim 6 where said arylaminobenzoate derivative is a benzamide derivative having a neutral charge.

8. The method of claim 7 where the benzamide derivative is NPPB-Am.

9. The method of claim 8 where said NPPB-Am is a pure chloride channel agonists.

10. The method of claim 8 where said NPPB-Am stimulates a rate of calcium channel opening without altering chloride channel phosphorylation.

11. The method of claim 8 where said NPPB-Am stimulates a channel opening rate of said channel.

12. The method of claim 6 where said arylaminobenzoate derivative is a benzenesulfonamide derivative.

13. The method of claim 12 where said benzenesulfonamide derivative is NPPB-sulf.

14. The method of claim 1 where the compound is curcumin.

15. The method of claim 14 where said curcumin is a pure chloride channel agonists.

16. The method of claim 14 where said curcumin stimulates a the rate of calcium channel opening without altering chloride channel phosphorylation.

17. The method of claim 14 where said curcumin stimulates a channel opening rate of said channel.

18. The method of claim 1 where the cystic fibrosis transmembrane conductance regulator is sub-optimally stimulated, a ΔF508 mutant or a G551D mutant.

19. The method of claim 1 where the compound is administered orally.

20. The method of claim 1 where said compound is administered parenterally.

21. The method of claim 1 where said compound is administered by inhalation.

22. The method of claim 1 where said compound is administered by intranasal inhalation of intrapulmonary administration.

23. The method of claim 1 where said compound is administered transdermally.

24. The method of claim 1 where said method further comprising administering a second compound.

25. The method of claim 1 where said treatment results in an increased hydration of a mucous lining in a lung.

26. A method for treating a disease state characterized by a chloride channel having sub-optimal activity in a subject in need of such treatment, said method comprising the step of administering to said subject a therapeutically effective amount of a compound capable of stimulating the activity of the chloride channel having sub-optimal activity.

27. The method of claim 26 where said compound is an arylaminobenzoate, an arylaminobenzoate derivative or curcumin.

28. The method of claim 26 where said compound is NPPB, NPPB-Am, curcumin or NPPB-sulf.

29-51. (canceled)

52. A method for increasing the activity of a chloride channel having sub-optimal activity, said method comprising the step of administering a compound capable of stimulating the activity of the chloride channel having sub-optimal activity.

53. The method of claim 52 where said compound is an arylaminobenzoate, an arylaminobenzoate derivative or curcumin.

54. The method of claim 52 53 where said compound is NPPB, NPPB-Am curcumin or NPPB-sulf.

55-76. (canceled)
